(19) United States
(12) Patent Application Publication
(10) Pub. No.: US 2022/0334116 A1
(43) Pub. Date: Oct. 20, 2022

(54) METHODS FOR MONITORING OR PREDICTING RESPONSE TO IMMUNOTHERAPIES FOR GYNECOLOGIC CANCER

(71) Applicant: ARIZONA BOARD OF REGENTS ON BEHALF OF THE UNIVERSITY OF ARIZONA, Tucson, AZ (US)

(72) Inventors: Melissa M. Herbst-Kralovetz, Phoenix, AZ (US); Pawel Laniewski, Phoenix, AZ (US)

(21) Appl. No.: 17/633,720

(22) PCT Filed: Aug. 10, 2020
(86) PCT No.: PCT/US20/45665

§ 371 Date: Feb. 8, 2022

Related U.S. Application Data
(60) Provisional application No. 62/884,815, filed on Aug. 9, 2019.

(51) Int. Cl. G01N 33/574; G01N 33/569

(52) U.S. Cl. CPC G01N 33/57411; G01N 33/56911

(57) ABSTRACT

Non-invasive methods of evaluating a broad range of immune checkpoint biomarkers as well as other characteristics such as disease status, pH, Laciohacillm abundance, inflammation, etc., in the local cervicovaginal microenvironment. The immune checkpoint biomarkers and other characteristics may be used to monitor disease status and responses to therapies, stratify patients into groups of predicted non responders and responders with respect to a particular therapy, predicting whether a patient may have toxicity issues with a particular therapy, etc. The methods herein may also help distinguish between different biological processes, such as cancer and dysplasia.

### CROSS-REFERENCES TO RELATED APPLICATIONS

1011 This application claims benefit of U.S. Provisional Application No. 62/884,815 filed Aug. 9, 2019, the specification(s) of which is/are incorporated herein in their entirety by reference.

### FIELD OF THE INVENTION

The present invention relates to methods for monitoring cancers effecting women (e.g., cervical cancer), for example monitoring responses to particular cervical cancer therapies such as immunotherapies, as well as stratifying patients into categories such as non-responders and responders to a particular therapy or those susceptible to toxicity. The methods feature detecting particular biomarkers, such as immune checkpoints and/or microbiota, using the local microenvironment instead of blood samples. The present invention is not limited to gynecologic cancers.

### BACKGROUND OF THE INVENTION

Cervical cancer is the most common human papillomavirus (HPV)-related cancer and the fourth most common cancer in women worldwide with estimated 570,000 new cases and 311,000 deaths in 2018. Over the last few decades, widespread use of screening methods, such as Papanicolaou test (cervical cytology) and HPV-based molecular tests resulted in a dramatic decline in the number of deaths related to cervical cancer in many high-income countries. However, in lower income countries, the majority of women are diagnosed with advanced or metastatic carcinoma, which contributes to high mortality related to cervical cancer. In addition, the death rates in the United States did not significantly change since 2007 (2.2 per 100,000 women) (https://www.cdc.gov/cancer/uscs/). The American Cancer Society estimates 13,170 new cases of cervical cancer cases and 4,250 deaths in the United States in 2019. The majority of these new cervical cancer cases occurred among women who had never or rarely been screened. It is also worth noting that 90 percent of cervical cancer cases could be also preventable through routine HPV vaccination for adolescent males and females. Yet, the HPV vaccination coverage in the United States remains drastically lower (43% in 2017) compared to other recommended childhood vaccinations [e.g. 91% for measles, mumps and rubella (MMR) vaccine].

Standard treatments of cervical cancer include surgery, chemoradiation or a combination of both, depending on the stage of cancer. Despite advances in screening and prevention, the five-year overall survival for all stages of cervical cancer is 68%, whereas the five-year overall survival for advanced cervical cancer is only 15%. Introduction of an anti-angiogenic agent (bevacizumab) to chemotherapy increased overall survival from 13 to 17 months for patients with persistent, recurrent and metastatic cancer. Yet, there is an urgent need to improve therapeutic outcomes, particularly for advanced or relapsed disease.

Recently, immune checkpoint inhibitors have emerged as a promising strategy for the treatment of advanced solid tumors, including cervical cancer. Indeed, in 2018, the Food and Drug Administration approved pembrolizumab, a monoclonal antibody targeting the programmed cell death protein 1 (PD-1), for advanced cervical cancer with disease progression during or after chemotherapy. Other immune checkpoint inhibitors currently under investigation for cervical cancer treatment include antibodies targeting programmed cell death ligand 1 (PD-L1) or cytotoxic T-lymphocyte antigen 4 (CTLA-4). However, the results from recent phase II clinical trials revealed that the overall response to anti-PD-1 or anti-PD-L1 therapies was low, ranging from 12.5 to 26%. These clinical studies also demonstrated that the overall response rate was independent of PD-L1 expression, HPV status or number of previous therapies. These findings highlight that predictive biomarkers for patient responsiveness to immunotherapies are still urgently needed.

Vaginal microbiota in the majority of healthy premenopausal women is dominated by Lactobacillus species (L. crispatus, L. gasseri, L. jenseni, or L. iners), which protects the host against sexually transmitted infections, such as HPV. To date, multiple epidemiological studies consistently demonstrated a decrease in Lactobacillus dominance and an increase in dysbiotic communities, characterized by overgrowth of diverse anaerobic microorganisms, in women with cervical dysplasia and cancer. Two recent meta-analyses of available data strongly support a role of the vaginal microbiota in HPV persistence and cervical disease progression.

Notably, the present invention demonstrated that host factors in cervicovaginal lavages, including circulating cancer biomarkers, depend on genital inflammation and the vaginal microbiota composition. To better understand the biological mechanisms of cervical neoplastic disease, immune checkpoint protein profiles herein were investigated in cervicovaginal lavages collected from women across cervical carcinogenesis in the context of the vaginal microbiota and genital inflammation. This integrated approach uncovered the multifaceted interactions in the local microenvironment involving bacteria and mediators regulating host defense activation, which may be translated in future studies related to disease progression and/or efficacy of immunotherapies.

### BRIEF SUMMARY OF THE INVENTION

It is an objective of the present invention to provide method of monitoring disease status and responses to therapies, stratifying patients into groups of predicted non-responders and responders with respect to a particular therapy, predicting whether a patient may have toxicity issues with a particular therapy, etc. using samples collected from the local cervicovaginal microenvironment, as specified in the independent claims. Embodiments of the invention are given in the dependent claims. Embodiments of the present invention can be freely combined with each other if they are not mutually exclusive.

The present invention features methods of diagnosis invasive cervical carcinoma (ICC) in a patient. In some embodiments, the method comprises determining the patient's levels of two or more immune checkpoint proteins. In some embodiments, the checkpoint proteins are determined by obtaining a cervicovaginal lavage (CVL) sample from the patient and measuring the levels of two or more checkpoint proteins in the sample obtained. In some embodiments, if the patient has levels of at least two or more immune checkpoint proteins above a predetermined threshold then the patient is diagnosed with ICC. In some embodiments if the patient has levels of at least two or more immune checkpoint proteins below a predetermined threshold then the patient is diagnosed with dysplasia. In some embodiments, the predetermined threshold is the concentration over a defined threshold or a fold change or specific concentration in pg/ml).

The present invention also features methods of predicting a response to a therapy for treating invasive cervical carcinoma (ICC). In some embodiments, the method comprises obtaining a cervicovaginal lavage (CVL) sample and treating said sample to detect levels of at least two biomarkers from a group consisting of duster of differentiation (CD) 40, T-cell immunoglobulin and mucin domain-containing 3 (TIM-3), CD27, programmed cell death protein ligand 1 (PD-L1), lymphocyte activation gene 3 (LAG-3), toll-like receptor 2 (TLR-2), herpesvirus entry mediator (HVEM), CD28, cytotoxic T-lymphocyte antigen 4 (CTLA-4), glucocorticoid-induced tumor necrosis factor receptor-related protein (GITR), GITR ligand (GITRL), CD86, B- and T-lymphocyte attenuator (BTLA), inducible T-cell co-stimulator (ICOS), CD80, Lactobacillus abundance, and inflammation. The levels of at least two blornarkers are indicative of a particular state of ICC and indicate whether the response to a therapy will be positive or negative.

Additionally, the present invention may also feature a method of obtaining a cervicovaginal lavage (CVL) sample from a patient and producing a profile. In some embodiments the CVL sample profile is produced by detecting at least two or more immune checkpoint biomarkers and detecting the microbiota population. In some embodiments, the CVL profile produced is analysed.

One of the unique and inventive technical features of the present invention allows for a method to monitor disease status and response to therapies, stratify patients into groups of predicted non-responders and responders with respect to a particular therapy, predict whether a patient may have toxicity issues with a particular therapy, etc. using samples collected from local microenvironments (i.e. using cervicovaginal lavage (CVL) and vaginal swabs). Without wishing to limit the invention to any theory or mechanism, it is believed that the technical feature of the present invention advantageously provides for a minimally-invasive, low cost, easy means of evaluating a broad range of immune checkpoint biomarkers as well as other characteristics such as disease status, pH, Lactobacillus abundance, Inflammation, etc. Further, the present invention is not limited to a small subset of commonly evaluated checkpoint biomarkers and instead includes a large number of biomarkers (and other microenvironment characteristics) that are not normally examined. None of the presently known prior references or work has the unique inventive technical feature of the present invention.

Furthermore, the prior references teach away from the present invention. For example, previously measuring checkpoint biomarkers was done with blood test or biopsy, which can be invasive. Additionally, only a small subset of checkpoint biomarkers were even evaluated. Contrary to the prior arts, the present invention was able to measure useful biomarkers from cervicovaginal lavage (CVL) samples. Further still, the inventive technical features of the present invention contributed to a surprising result. For example, inventors surprisingly discovered that the microbiome in the cervicovaginal microenvironment can drive the level of a particular biomarker of interest. For example, PDL-1 and LAG3 were both correlated/associated with non-Lactobacillus dominance. Furthermore. TLR2 was surprisingly correlated with both Lactobacillus abundance as well as inflammation.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows that cervical cancer patients exhibit distinct local immune checkpoint profiles, Immune checkpoint proteins are present in the local cervicovaginal microenvironment. Local protein profiles are distinct in cervical cancer patients compared to precancerous and control groups. Principal component analysis (PCA) of immune checkpoint protein profiles displayed along the first two principal components (PC), with each point representing a single sample (a single patient) colored according to disease group (n=78). Box-and-whiskers plots shown along each PC axis represent the median and interquartile range with whiskers between 10^{th} and 90^{th} percentiles and indicate the distribution of samples along the given axis; dots indicate outliers. P value for the first two components was calculated using MANOVA, whereas P values for individual components were assessed using ANOVA

FIGS. 2A-2B show that local levels of immune checkpoint proteins are elevated in cervical cancer. Cervical cancer patients exhibit significantly elevated levels of immune checkpoint proteins from inhibitory (FIG. 2A) and co-stimulatory (FIG. 28) pathways in cervicovaginal lavages compared to other groups. Violin plots show distribution of protein levels in ICC (n=10) and Ctrl HPV− groups (n=18). Dots indicate individual values for each sample and horizontal solid and dashed lines indicate median and first and third quartiles, respectively. Immune checkpoint proteins that form functional complexes were grouped together. P values were calculated using linear mixed effects models where group was the fixed effect and replicate was the random effect with Tukey adjustment. Immune checkpoint that reached significant differences (P<0.05) between Ctrl HPV− and ICC are depicted in green and red (as well as a *), whereas immune checkpoint proteins that did not reach significant differences are depicted in gray. Levels of immune checkpoint proteins among all patient groups are included in FIG. 9.

FIG. 3 shows that CD40, TIM-3, and CD27 discriminate cervical cancer from other groups. CD40 is an excellent discriminator and TIM-3, CD27 are good discriminators for cervical cancer when compared to controls and precancerous dysplasia. The receiver operating characteristics (ROC) analysis compares cervical cancer group (n=10) to Ctrl HPV− (n=18), Ctrl HPV+(n=11), LSIL (n=12) or HSIL (n=27) groups. ROC curves indicate specificity (x axis) and 1-sensitivity (y axis). Immune checkpoint proteins with the area under curve (AUC) greater than 0.7, 0.8 or 0.9 serve as fair, good or excellent discriminators, respectively. Only immune checkpoint proteins with AUC>0.7 are depicted. ROC curves for all tested immune checkpoint proteins are included in FIG. 10.

FIG. 4 shows a correlation network of cervicovaginal immune checkpoint proteins. Levels of immune checkpoint proteins in the local microenvironment are strongly correlated to other immune checkpoint proteins. Correlation matrix of immune checkpoint proteins in the cervicovaginal lavages among all the patients (n=78). Correlation coefficient (p) was calculated using Spearman's rank correlation analysis. Hierarchical clustering of correlation coefficients was performed using ClustVis based on Euclidean distance and average linkage cluster algorithm. Purple- and yellow-shaded squares on the heatmap indicate positive (1) and negative (−1) correlation, respectively. Clusters of proteins that strongly correlate to each other are depicted with green outlines. A bar below each dendrogram shows immune pathways utilized by immune checkpoint proteins. Significant correlations (P<0.05) are indicated with black circles (•).

FIGS. 5A-5B show that key checkpoint proteins correlate with Lactobacillus and genital inflammation. Scatterplots shows correlations of immune checkpoint proteins with Lactobacillus abundance (x axis) or genital inflammatory scores (y axis). Spearman's correlation coefficients (ρ) were calculated using levels of immune checkpoint proteins for all samples (n=78) (FIG. 5A) or without cancer samples (n=68) (FIG. 5B). Lactobacifus abundance was determined by 16S rRNA gene sequencing. Levels of seven cytokines (IL-1α, IL-1β, IL-8, MIP-1β, MIP-3a, RANTES, TNFα) were evaluated in CVLs and the patients were assigned a genital inflammatory score (0-7) based on whether the level of each cytokine was in the upper quartile. Significant correlations (P<0.05) between immune checkpoint proteins and genital inflammatory scores, Lactobacillus abundance or both are indicated with red, green and blue circles, respectively.

FIG. 6 shows PD-L1, LAG-3, and TLR2 correlate to the most abundant vaginal bacterial species. PD-L1 and LAG-3 negatively correlated to Lactobacillus species and positively correlated to dysbiotic bacteria, whereas TLR2 negatively correlated to dysbiotic bacteria and positively correlated to lactobacilli. Spearman correlation coefficients (ρ) were calculated using levels of immune checkpoint proteins for all samples (n=78) and relative abundances/levels of vaginal bacterial taxa. Relative abundances of most prevalent vaginal genera (i.e. Lactobacillus, Gardnerella, Sneathia, Prevotella, Atopobium, Megasphaera and Streptococcus) were determined by 16S rRNA gene sequencing. Relative levels of vaginal Lactobacillus species (L. crispatus, L. gasseri, L. jensenii and L. iners) were determined by quantitative real-time PCR assays. P values are indicated with asterisks (***P<0.001, **P<0.01, *P<0.05).

FIGS. 7A-7B show a complex host-microbe network in the cervicovaginal microenvironment. Venn (FIG. 7A) and network (FIG. 7B) diagrams summarize the results of this study and depict immune checkpoint proteins significantly elevated in patients with invasive cervical carcinoma when compared to HPV-negative controls (indicated in pink); immune checkpoint proteins significantly correlated to genital inflammatory scores (indicated in purple); and immune checkpoint proteins significantly correlated to vaginal microbiota structure (indicated in green). The network diagram also shows correlations of immune checkpoint proteins to other immune checkpoint proteins. Solid and dotted lines indicate positive or negative relationships, respectively. TLR2 was the only gene to have a negative correlation. TLR2 was lower in dysbiotic bacteria but was higher in genital inflammation.

FIG. 8 shows the contribution of immune checkpoint proteins in the principal component analysis (PCA). Stacked bar plots show contribution of each immune checkpoint protein to principal 1q principal component (PC2), which explains 49.7% and 20.7% of the variance in the data, respectively. CD80, LAG-3 and PD-L1 levels contribute mostly to PC1 and CD40. HVEM and TLR2 levels contribute mostly to PC2, whereas the other immune checkpoint proteins contribute to both PC1 and PC2.

FIG. 9 shows the level of immune checkpoint proteins in cervicovaginal lavages in Ctrl HPV−, Ctrl HPV+, LSIL, HSIL and ICC groups. Scatter plots show distribution of protein levels across the groups: healthy HPV-negative controls (Ctrl HPV−), HPV-positive controls (Ctrl HPV+), low-grade squamous intraepithelial lesion (LSIL), high-grade squamous intraepithelial lesion (HSIL) and invasive cervical carcinoma (ICC). Dots indicate individual values for each sample and horizontal solid and dashed lines indicate median and first and third quartiles, respectively. P values were calculated using linear mixed effects models where group was the fixed effect and replicate was the random effect with Tukey adjustment. P values are indicated with asterisks (****P<0.0001, ***P<0.001, **P<0.01, *P<0.05).

FIG. 10 shows the discrimination capacities of immune checkpoint proteins in cervicovaginal lavages for cervical cancer. The receiver operating characteristics (ROC) analysis comparing invasive cervical carcinoma (ICC) to healthy HPV-negative controls (Ctrl HPV−). ROC curves indicate specificity (x axis) and 1—sensitivity (y axis). Immune checkpoint proteins with the area under curve (AUC) greater than 0.6, 0.7, 0.8 or 0.9 serve as poor, fair, good or excellent discriminators, respectively. ROC plots are arranged in a decreasing order of AUC values.

FIGS. 11A-11B show the correlation of immune checkpoint proteins to other immune checkpoint proteins in cervicovaginal lavages among all the patients. Correlation coefficients (ρ) were calculated using Spearman's rank correlation analysis. Heat maps shows Spearman's rank correlation coefficients (FIG. 11A) or P values (FIG. 11B). Red and blue squares indicate positive or negative correlations, respectively, whereas green squares depict different ranges of P values.

### DETAILED DESCRIPTION OF THE INVENTION

In some embodiments, the present invention describes evaluating a broad range of immune checkpoint proteins in the local cervicovaginal microenvironment to illustrate features (e.g., disease status, vaginal pH, Lactobacillus abundance, genital inflammation, etc.) that are associated with local changes in the pattern and quantity of these targets. In other embodiments, the methods feature, for example, detecting particular biomarkers, such as immune checkpoint biomarkers, and/or other characteristics of the local environment such as microbiota and pH, using the local microenvironment instead of blood samples. For example, immune checkpoint biomarkers are present in the local environment and can be readily quantified in cervicovaginal lavage samples.

Several checkpoint biomarkers are highly associated with disease status and features of the local microenvironment, such as genital inflammation and Lactobacillus abundance. In some embodiments, the present invention provides methods for understanding the contribution of local immune checkpoint biomarkers that may improve patient outcomes by helping to predict therapeutic response and/or toxicity.

The present invention is not limited to gynecologic cancers. In some embodiments the present invention may include cancers (such as but not limited to ovarian cancer, cervical cancer, endometrial cancer, breast cancer, gastric cancer, colorectal cancer, lung cancer) and other gynecologic conditions (e.g., endometriosis, adenomyosis, PCOS, chronic pelvic pain. In some embodiment the present invention may include cancer affecting women.

The term “cancer” refers to any physiological condition in mammals characterized by unregulated cell growth. Cancers described herein include solid tumors. A“solid tumor” or “tumor” refers to a lesion and neoplastic cell growth and proliferation, whether malignant or benign, and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

The term “dysplasia” may refer to when healthy cells undergo abnormal changes within tissues or organs, and it is considered as a pre-cancerous disease state. In some embodiment dysplasia may progress and become cancer. In other embodiments, dysplasia may regress. The term “pre-cancerous disease state” may refer to a condition or lesion involving abnormal cells which are associated with an increased risk of developing into cancer. In some embodiments, the progression of normal cells to precancerous cells and towards invasive carcinoma may involve oncogenes, inflammation, and multiple somatic mutations that initiate the malignant transformation, activation, and clonal expansion of stem cells.

As used herein “immune checkpoint biomarkers” may refer to a group of proteins that regulate the immune system and play a crucial role in self-tolerance as well as in anti-cancer immune response. In some embodiments, the level of these proteins can be measured using antibody-based protein assays such as but not limited to ELISA and cytometric bead arrays. In some embodiments, cervicovaginal lavage (CVL) samples are analysed using antibody-based protein assays to detect and measure immune checkpoint biomarker proteins. In some embodiments, immune checkpoint biomarkers may refer to but are not limited to cluster of differentiation (CD) 40, T-cell immunoglobulin and mucin domain-containing 3 (TIM-3), CD27, programmed cell death protein ligand 1 (PD-L1), lymphocyte activation gene 3 (LAG-3), toll-like receptor 2 (TLR-2), herpesvirus entry mediator (HVEM), CD28, cytotoxic T-lymphocyte antigen 4 (CTLA-4), glucocorticoid-induced tumor necrosis factor receptor-related protein (GITR), GITR ligand (GITRL), or CD86.

As used herein “inflammation” may refer to a localized physical condition in which part of the body becomes reddened, and swollen, especially as a reaction to injury or infection or a state of locally elevated levels of pro-inflammatory cytokines and chemokines. In some embodiments inflammation may be short-lived (acute) or long-live (chronic). In some embodiments, inflammation is measured by evaluation of levels of cytokines in CVL samples. In some embodiments, inflammation is measured by evaluation of levels of seven cytokines in CVL samples which may include but are not limited to interleukin 1 alpha (IL-1α), interleukin 1 beta (IL-1β), interleukin 8 (IL-8), macrophage inflammatory proteins 1β (MIP-1β). C-C motif chemokine ligand 20 (CCL20), regulated on activation, normal T cell expressed and secreted (RANTES), tumor necrosis factor (TNFα). In some embodiments, markers of inflammation may include but are not limited to IL-1a, IL-1b, IL-8, MIP-1b, CCL20, RANTES, TNFa. In some embodiments, patients am assigned a genital inflammatory score (0-7) based on whether the level of each cytokine was in the upper quartile. In some embodiments, Inflammatory scores 0, 1-4, 5-7 define none, low and high inflammation, respectively.

As used herein “Lactobacillus abundance” may refer to the relative abundance of various Lactobacillus species that may include but is not limited to L. crispatus, L. gasseri, L. jensenii, L. iners measured by next generation sequencing methods or PCR-based assays.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder or condition described herein. In certain instances, the term patient refers to a human.

As used herein, the term “healthy control” may refer to a subject without cancer, dysplasia and genital infection (e.g., HPV).

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The terms “manage,” “managing,” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

The terms “regress,” “regressing,” and “regression” may refer to a decrease in the size of a tumor or in the extent of cancer in the body. In some embodiments, “regression” may refer to a decrease in severity of the disease and/or decrease in the size of a tumor. In some embodiments, regression may generally refer to lighter symptoms without the disease completely disappearing. In certain cases, the beneficial effects that a subject derives from a prophylactic or therapeutic agent do not result in a cure of the disease or disorder. In some embodiments, symptoms of the disease may return.

The term “effective amount” as used herein refers to the amount of a therapy (e.g., an anti-cancer agent or radiation therapy provided herein) which is sufficient to reduce and/or ameliorate the severity and/or duration of a given disease, disorder or condition and/or a symptom related thereto. This term also encompasses an amount necessary for the reduction or amelioration of the advancement or progression of a given disease (e.g., cancer), disorder or condition, reduction or amelioration of the recurrence, development or onset of a given disease, disorder or condition, and/or to improve or enhance the prophylactic or therapeutic effect(s) of another therapy. In some embodiments. “effective amount” as used herein also refers to the amount of therapy provided herein to achieve a specified result.

As used herein, and unless otherwise specified, the term “therapeutically effective amount” of an anti-cancer agent or a radiation therapy described herein is an amount sufficient to provide a therapeutic benefit in the treatment or management of a cancer, or to delay or minimize one or more symptoms associated with the presence of the cancer. A therapeutically effective amount of an anti-cancer agent described herein, or a radiation therapy described herein means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent.

A therapy is any protocol, method and/or agent that can be used in the prevention, management, treatment and/or amelioration of a given disease, disorder or condition. In certain embodiments, the terms “therapies” and “therapy” refer to a drug therapy, biological therapy, supportive therapy, radiation therapy, and/or other therapies useful in the prevention, management, treatment and/or amelioration of a given disease, disorder or condition known to one of skill in the art such as medical personnel.

The term “anti-cancer agent” is used in accordance with its plain ordinary meaning and refers to a composition having anti-neoplastic properties or the ability to inhibit the growth or proliferation of cells. In certain embodiments, an anti-cancer agent is chemotherapeutic. In certain embodiments, an anti-cancer agent is an agent identified herein having utility in methods of treating cancer. In certain embodiments, an anti-cancer agent is an agent approved by the FDA or similar regulatory agency of a country other than the USA, for treating cancer.

The term “chemotherapeutic” or “chemotherapeutic agent” is used in accordance with its plain ordinary meaning and refers to a chemical composition or compound having anti-neoplastic properties or the ability to inhibit the growth or proliferation of cells. “Chemotherapy” or “cancer therapy” refers to a therapy or regimen that includes administration of a combination, chemotherapeutic, or anti-cancer agent.

The term “radiation therapy” is used in accordance with its plain ordinary meaning and refers to the medical use of radiation in the treatment of cancer. Preferably, the medical use of radiation in the treatment of cancer results in the killing of cancer cells in the subject.

The term “immunotherapy” is used in accordance with its plain ordinary meaning and refers to the medical use of activating or suppressing the immune system to treat cancer. Preferably, the medical use of immunotherapy in the treatment of cancers refers to the activation of the immune cells within the body to destroy abnormal/cancer cells in the subject.

The present invention features methods of diagnosing invasive cervical carcinoma (ICC) in a patient. In some embodiments, the method comprises determining the patient's levels of two or more immune checkpoint proteins. In some embodiments, the checkpoint proteins are determined by obtaining a cervicovaginal lavage (CVL) sample from the patient and measuring the levels of two or more checkpoint proteins in the sample obtained. In some embodiments, if the patient has levels of at least two or more immune checkpoint proteins above a predetermined threshold then the patient is diagnosed with ICC. In some embodiments if the patient has levels of at least two or more immune checkpoint proteins below a predetermined threshold then the patient is diagnosed with dysplasia. In some embodiments, the predetermined threshold is the concentration over a defined threshold or a fold change or specific concentration in pg/ml).

In some embodiments, the immune checkpoint protein may be cluster of differentiation 40 (CD40). In some embodiments, a CLV sample may have a CD40 range of 200-800 pg/ml for a patient with cancer. In some embodiments, CD40 may be 100 pg/ml in a patient with cancer. In some embodiments, CD40 may be 200 pg/ml in a patient with cancer. In some embodiments, CD40 may be 300 pg/ml in a patient with cancer. In some embodiments, CD40 may be 400 pg/ml in a patient with cancer. In some embodiments, CD40 may be 500 pg/ml in a patient with cancer. In some embodiments, CD40 may be 600 pg/ml in a patient with cancer. In some embodiments, CD40 may be 700 pg/ml in a patient with cancer. In some embodiments, CD40 may be 800 pg/ml in a patient with cancer. In some embodiments, CD40 may be 900 pg/ml in a patient with cancer. In some embodiments, the levels of CD40 in a patient with cancer may be 100 pg/ml, 200 pg/ml, 300 pg/ml, 400 pg/ml, 500 pg/ml, 600 pg/ml, 700 pg/ml, 800 pg/ml or 900 pg/ml. In some embodiments, a CLV sample may have a CD40 range of 8-80 pg/ml for a healthy control. In some embodiments, CD40 may be 5 pg/ml in a healthy control. In some embodiments, CD40 may be 10 pg/ml in a healthy control. In some embodiments, CD40 may be 20 pg/ml in a healthy control. In some embodiments, CD40 may be 30 pg/ml in a healthy control. In some embodiments, CD40 may be 40 pg/ml in a healthy control. In some embodiments, CD40 may be 50 pg/ml in a healthy control. In some embodiments, CD40 may be 60 pg/ml in a healthy control. In some embodiments, CD40 may be 70 pg/ml in a healthy control. In some embodiments, CD40 may be 80 pg/ml in a healthy control. In some embodiments, CD40 may be 90 pg/ml in a healthy control. In some embodiments, the levels of CD40 in a healthy control may be 5 pg/ml, 10 pg/ml, 20 pg/ml, 30 pg/ml, 40 pg/ml, 50 pg/ml, 60 pg/ml, 70 pg/ml, 80 pg/ml or 90 pg/ml.

In some embodiments, the immune checkpoint protein may be cluster of differentiation 27 (CD27). In some embodiments, a CLV sample may have a CD27 range of 20-500 pg/ml for a patient with cancer. In some embodiments, CD27 may be 10 pg/ml in a patient with cancer. In some embodiments, CD27 may be 20 pg/ml in a patient with cancer. In some embodiments, CD27 may be 30 pg/ml in a patient with cancer. In some embodiments, CD27 may be 40 pg/ml in a patient with cancer. In some embodiments, CD27 may be 50 pg/ml in a patient with cancer. In some embodiments. CD27 may be 60 pg/md in a patient with cancer. In some embodiments, CD27 may be 70 pg/ml in a patient with cancer. In some embodiments, CD27 may be 80 pg/ml in a patient with cancer. In some embodiments, CD27 may be 90 pg/md in a patient with cancer. In some embodiments, CD27 may be 100 pg/ml in a patient with cancer. In some embodiments. CD27 may be 150 pg/ml in a patient with cancer. In some embodiments, CD27 may be 200 pg/ml in a patient with cancer. In some embodiments, CD27 may be 250 pg/i in a patient with cancer. In some embodiments, CD27 may be 300 pg/ml in a patient with cancer. In some embodiments, CD27 may be 350 pg/ml in a patient with cancer. In some embodiments, CD27 may be 400 pg/ml in a patient with cancer. In some embodiments, CD27 may be 450 pg/ml in a patient with cancer. In some embodiments, CD27 may be 500 pg/ml in a patient with cancer. In some embodiments, CD27 may be 550 pg/ml in a patient with cancer. In some embodiments, the levels of CD27 in a patient with cancer may be 10 pg/ml, 20 pg/ml, 30 pg/ml, 40 pg/ml, 50 pg/ml, 60 pg/ml, 70 pg/ml, 80 pg/ml or 90 pg/ml, 100 pg/ml, 150 pg/ml, 200 pg/mt, 250 pg/ml, 300 pg/ml, 350 pg/ml, 400 pg/ml 450 pg/ml, 500 pg/ml, or 550 pg/ml. In some embodiments, a CLV sample may have a CD27 range of 1-20 pg/ml for a healthy control. In some embodiments, CD27 may be 0.5 pg/ml in a healthy control. In some embodiments, CD27 may be 1 pg/ml in a healthy control. In some embodiments, CD27 may be 5 pg/ml in a healthy control. In some embodiments, CD27 may be 10 pg/ml in a healthy control. In some embodiments, CD27 may be 15 pg/ml in a healthy control. In some embodiments, CD27 may be 20 pg/ml in a healthy control. In some embodiments, CD27 may be 25 pg/ml in a healthy control. In some embodiments, the levels of CD27 in a healthy control may be 0.5 pg/ml, 1 pg/ml, 5 pg/ml, 10 pg/ml, 15 pg/ml, 20 pg/ml, and 25 pg/ml.

In some embodiments, the immune checkpoint protein may be T-cell immunoglobulin and mucin domain-containing 3 (TIM-3). In some embodiments, a CLV sample may have a TIM-3 range of 20-300 pg/ml for a patient with cancer. In some embodiments, TIM-3 may be 10 pg/ml in a patient with cancer. In some embodiments, TIM-3 may be 20 pg/ml in a patient with cancer. In some embodiments, TIM-3 may be 30 pg/ml in a patient with cancer. In some embodiments, TIM-3 may be 40 pg/ml in a patient with cancer. In some embodiments, TIM-3 may be 50 pg/ml in a patient with cancer. In some embodiments, TIM-3 may be 80 pg/ml in a patient with cancer. In some embodiments, TIM-3 may be 70 pg/ml in a patient with cancer. In some embodiments, TIM-3 may be 80 pg/ml in a patient with cancer. In some embodiments, TIM-3 may be 90 pg/ml in a patient with cancer. In some embodiments, TIM-3 may be 100 pg/ml in a patient with cancer. In some embodiments, TIM-3 may be 150 pg/m in a patient with cancer. In some embodiments, TIM-3 may be 200 pg/ml in a patient with cancer. In some embodiments, TIM-3 may be 250 pg/ml in a patient with cancer. In some embodiments, TIM-3 may be 300 pg/ml in a patient with cancer. In some embodiments, TIM-3 may be 350 pg/rI in a patient with cancer. In some embodiments, the levels of TIM-3 in a patient with cancer may be 10 pg/ml, 20 pg/ml, 30 pg/ml, 40 pg/ml, 50 pg/ml, 60 pg/mW, 70 pg/ml, 80 pg/ml or 90 pg/ml, 100 pg/ml, 150 pg/ml, 200 pg/ml, 250 pg/ml, 300 pg/ml, or 350 pg/ml. In some embodiments, a CLV sample may have a TIM-3 range of 0.3-15 pg/ml for a healthy control. In some embodiments, TIM-3 may be 0.1 pg/ml in a healthy control. In some embodiments, TIM-3 may be 0.5 pg/ml in a healthy control. In some embodiments, TIM-3 may be 1 pg/ml in a healthy control. In some embodiments, TIM-3 may be 5 pg/ml in a healthy control. In some embodiments, TIM-3 may be 10 pg/ml in a healthy control. In some embodiments, TIM-3 may be 15 pg/ml in a healthy control. In some embodiments, TIM-3 may be 20 pg/ml in a healthy control. In some embodiments, the levels of TIM-3 in a healthy control may be 0.1 pg/ml, 0.5 pg/ml, 1 pg/ml, 5 pg/ml, 10 pg/ml, 15 pg/ml, or 20 pg/ml.

The predetermined threshold may be an industry standard. The predetermined threshold may be a laboratory standard. As used herein “industry standard” may refer to concentration over a defined threshold or fold change or specific concentration in pg/ml. As used herein “laboratory standard” may refer to a range of concentrations of biomarkers in pg/ml. Additionally, as used herein, the “predetermined threshold” may refer to a range of concentrations in pg/ml in some embodiments, the predetermined threshold for CD40 may be 200-800 pg/ml for disease/cancer. In some embodiments, the predetermined threshold for CD27 may be 20-500 pg/ml disease/cancer. In some embodiments the predetermined threshold for TIM-3 may be 20-300 pg/ml disease/cancer. In some embodiments, algorithms (e.g., receiver operating characteristics, hierarchical clustering analysis, Random Forest analysis or neural network analysis) may be used to establish thresholds for levels of biomarkers. In some embodiments, algorithms may be used to establish thresholds for levels of biomarkers based on past datasets and findings.

The present invention also features methods of predicting a response to a therapy for treating invasive cervical carcinoma (ICC). In some embodiments, the method comprises obtaining a cervicovaginal lavage (CVL) sample and analysing said sample to detect levels of at least two biomarkers from a group consisting of cluster of differentiation (CD) 40, T-cell immunoglobulin and mucin domain-containing 3 (TIM-3), CD27, programmed cell death protein ligand 1 (PD-L1), lymphocyte activation gene 3 (LAG-3), toll-like receptor 2 (TLR-2), herpesvirus entry mediator (HVEM), CD28, cytotoxic T-lymphocyte antigen 4 (CTLA-4), glucocorticod-induced tumor necrosis factor receptor-related protein (GITR), GITR ligand (GITRL), CD86, B- and T-lymphocyte attenuator (BTLA), inducible T-cell co-stimulator (ICOS), CD80, Lactobacillus abundance, and inflammation. The levels of at least two biomarkers are indicative of a particular state of ICC and indicate whether the response to a therapy will be positive or negative.

In some embodiments, the method predicts a positive response to therapy. In some embodiments, a positive response may refer to regression of cancer or lack of progression or an increase in progression-free survival (PFS). As an example, in some embodiments, a positive response to a therapy is indicated by levels of both PD-1 and PD-L1 that are above a predetermined threshold. In some embodiments, a positive response to a therapy is indicated by levels of CD40 that are above a predetermined threshold. The present invention is not limited to the use of PD-L1, CD40, and PD-1. Further, the profile of the level of biomarkers may differ based on the therapy used. Algorithms may be used to establish thresholds for levels of biomarkers.

(In some embodiments, the method predicts a negative response to therapy. In some embodiments, a negative response may refer to a lack of response to therapy (cancer progression, death). As discussed above, the profile of the level of biomarkers may differ based on the therapy used. Algorithms may be used to establish thresholds for levels of biomarkers.

In some embodiments, a positive response to therapy may change the measured ranges of the immune checkpoint biomarkers as described herein. In some embodiments, a positive response to therapy may cause the ranges of the immune checkpoint markers described herein to decrease. In some embodiments, a positive response to therapy may cause the ranges of the immune checkpoint markers described herein to increase. In some embodiments, a positive response to therapy may cause the ranges of the immune checkpoint markers described herein to remain the same. In some embodiments, a negative response to a therapy may cause the ranges of the immune checkpoint biomarkers described herein to remain the same. In some embodiments, a negative response to therapy may change the measured ranges of the immune checkpoint biomarkers as described herein. In some embodiments, a negative response to therapy may cause the ranges of the immune checkpoint markers described herein to decrease. In some embodiments, a negative response to therapy may cause the ranges of the immune checkpoint markers described herein to increase.

In some embodiments, therapies may include but are not limited to anti-cancer therapeutics, chemotherapy, radiation therapy, or immunotherapy.

The present invention also features methods of predicting toxicity in a patient in response to a therapy for treating invasive cervical carcinoma (ICC). In some embodiments, the method comprises obtaining a cervicovaginal lavage (CVL) sample and analysing said sample to detect levels of at least two biomarkers selected from a group consisting of cluster of differentiation (CD) 40, T-cell immunoglobulin and mucin domain-containing 3 (TIM-3), CD27, programmed cell death protein ligand 1 (PD-L1), lymphocyte activation gene 3 (LAG-3), toll-ike receptor 2 (TLR-2), herpesvirus entry mediator (HVEM), CD28, cytotoxic T-lymphocyte antigen 4 (CTLA-4), glucocorticoid-induced tumor necrosis factor receptor-related protein (GITR), GITR ligand (GITRL), CD86, B- and T-lymphocyte attenuator (BTLA), inducible T-cell co-stimulator (ICOS), CD80, Lactobacillus abundance, and inflammation, wherein the levels of the biomarkers are indicative of a particular state of invasive cervical carcinoma.

As used herein “toxicity” in a patient may refer to an extent that something is harmful or poisonous to a patient.

(In some embodiments, a “particular state” of ICC may include different stages of cancer or may include different types of carcinoma (squamous cell carcinoma, adenocarcinoma). In some embodiments, the stages of cancer are defined by the international Federation of Gynecology and Obstetrics (FIGO) staging system.

The present invention also features methods of stratifying patients in a cohort into a group of responders and non-responders. In some embodiments, the responders are patients predicted to have a positive response to a therapy for treating invasive cervical carcinoma. In some embodiments, the non-responders are patients predicted to have no response or a negative response to a therapy for treating invasive cervical carcinoma.

The present invention also features methods of predicting a level of one or more of programmed cell death protein ligand 1 (PD-L1), T-cell immunoglobulin and mucin domain-containing 3 (TIM-3), cluster of differentiation (CD) 28, CD40, toll-like receptor 2 (TLR-2), herpesvirus entry mediator (HVEM). In some embodiments, the method comprises detecting inflammation in a cervicovaginal microenvironment, wherein a high level of inflammation is indicative of high levels of one or more of PD-1, TIM-3, CD28, CD40, TLR-2, and HVEM.

According to some embodiments, the invention features a method of predicting a level of one or more of programmed cell death protein ligand 1 (PD-L1), lymphocyte activation gene 3 (LAG-3), and toll-like receptor 2 (TLR-2). The method may comprise detecting a level of Lactobacillus abundance in a cervicovaginal microenvironment, and determining a level of one or both of PD-L1 and LAG-3 based on the level of Lactobacillus abundance.

The present invention also features methods of predicting a level of one or more of programmed cell death protein ligand 1 (PD-1), lymphocyte activation gene 3 (LAG-3), and toll-like receptor 2 (TLR-2). In some embodiments, the method comprises detecting Lactobacillus abundance in a cervicovaginal microenvironment, wherein a high level of Lactobacillus abundance is indicative of low levels of PD-L1 and LAG-3. In other embodiments, the method comprises detecting Lactobacillus abundance in a cervicovaginal microenvironment, wherein a high level of Lactobacillus abundance is indicative of high levels of TLR-2.

The present invention may also feature a method of obtaining a cervicovaginal lavage (CVL) sample from a patient and producing a profile. In some embodiments the CVL sample profile is produced by detecting at least two or more immune checkpoint biomarkers and detecting the microbiota population. In some embodiments, the CVL profile produced is analysed.

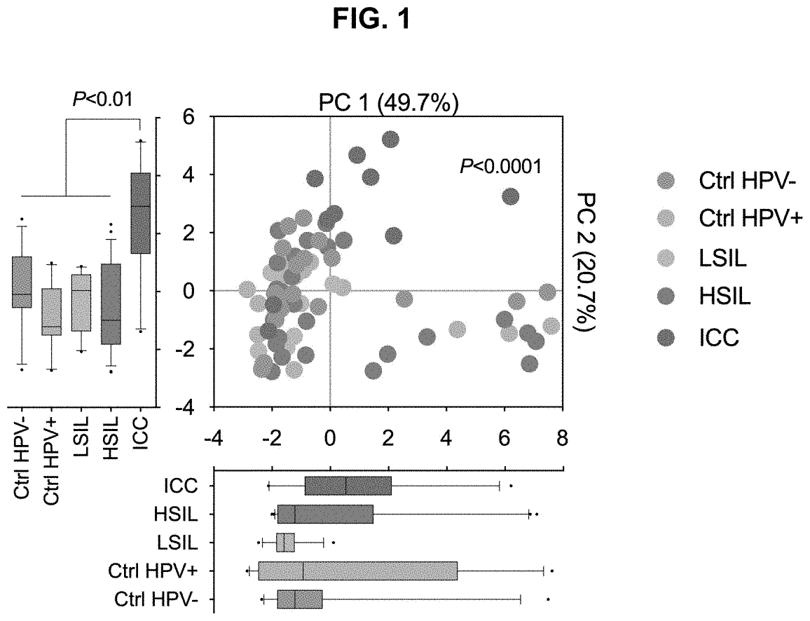

Referring to FIGS. 1-11B, sixteen immune checkpoint proteins were quantified in CVL using multiplex human checkpoint protein assay. The dissimilarities in the checkpoint protein datasets were analyzed using principal component analysis (PCA). The checkpoint protein discriminators for each patient group were identified using receiver operating characteristics (ROC) analysis and strength was determined by area under curve (AUC) values. Correlation with genital inflammatory scores, pH and Lactobacillus abundance was assessed using Spearman's rank correlation analysis. The statistical differences were tested using ANOVA or linear mixed effects models.

There was no significant difference among the groups in terms of age (P=0.48), ethnicity (P=0.15), or body mass index (P=0.97). Principal component analysis (PCA) demonstrated that invasive cervical carcinoma (ICC) patient samples duster from other groups (P<0.001). CD40 is an excellent discriminator (AUC 0.9-1.0) and CD27 and TIM-3 are good discriminators for invasive cervical carcinoma (ICC) (AUC 0.8-0.9) by ROC analysis. PD-1, TIM-3, CD28, CD40 and HVEM were positively associated with inflammation and LAG-3 was negatively associated with Lactobacillus abundance. PD-L1 was positively associated with inflammation and negatively associated with Lactobacillus abundance. TLR-2 was positively associated with both inflammation and Lactobacillus abundance. The study revealed that immune checkpoint proteins are present in CVL and several are highly associated with disease status and features of the local microenvironment (genital inflammation and Lactobacillus abundance). This is an initial step to understanding the contribution of local immune checkpoint proteins that may improve patient outcomes in the future by predicting therapeutic response and/or toxicity.

The present invention helps show the level to which genital immune checkpoint proteins associate with cervical disease status and/or features of the cervicovaginal microenvironment.

### EXAMPLE

The following is a non-limiting example of the present invention, it is to be understood that said example is not intended to limit the present invention in any way. Equivalents or substitutes are within the scope of the present invention.

### Clinical and Demographic Information:

Linear mixed effects model: The statistical differences in the concentration among the patient groups were tested using a linear mixed effects model where the group was a fixed effect and the replicate was the random effect. If the overall difference was significant (P<0.05), paired tests were performed with Tukey adjustment.

In this cross-sectional study, clinical samples were analyzed, i.e. cervicovaginal lavages (CVL) and vaginal swabs, collected from 78 premenopausal, non-pregnant, Arizonan women with and without HPV infection and cervical neoplasia. Women were recruited at three clinical sites in Phoenix, Ariz.: St. Joseph's Hospital and Medical Center, University of Arizona Cancer Center and Valleywise Health Medical Center. All participants provided informed written consent and all research and related activities involving human subjects were approved by the Institutional Review Boards at each participating site. Women were assigned to the following five groups: healthy HPV-negative controls (Ctrl HPV−; n=18), HPV-positive controls (Ctrl HPV+; n=11), women with low-grade intreepithelial lesions (LSIL; n=12), high-grade intraepithelial lesions (HSIL; n=27), and newly diagnosed invasive cervical carcinoma (ICC: n=10). Only patients with newly diagnosed ICC were included in the study. The classification to groups was based on the histology of biopsies, cytology (when histology was not available) and HPV genotyping. Detailed exclusion criteria were described in Laniewski, P et al. 2018 (Laniewski, P. et al. Linking cervicovaginal immune signatures, HPV and microbiota composition in cervical carcinogenesis in non-Hispanic and Hispanic women. Sci Rep 8, 7593, doi:10.1038/s41598-018-25879-7, 2018). Forty-seven percent of women were Hispanic, and 53% women were non-Hispanic. Sixty-eight percent of women were overweight or obese [body mass index (BMI>25] and the mean age across the groups was 38±8 years. However, there were no significant differences among the groups with regard to Hispanic, ethnicity (P=0.15), BMI (P=0.97) and age (P=0.46).

Cervicovaginal lavage (CVL) and vaginal swabs were collected by a physician and processed for microbiome and immunoproteome. A speculum was inserted without lubricant and two vaginal swabs were collected. The first swab was collected by swabbing the lateral walls of the mid vagina using an eSwab collection system containing Arnies transport medium (COPAN Diagnostics, Murrieta, Calif.). The second swab was used to measure vaginal pH using nitrazine paper and recorded by the clinician according to the manufacturer's instructions using a scale of 4.5-7.5. Following vaginal swab collection, CVLs were collected using 10 ml of sterile 0.9% saline (Teknova, Hollister, Calif.), immediately placed on ice and frozen at −80° C. for further analysis. DNA was extracted from vaginal swabs using the PowerSoil DNA Isolation Kit (MOBIO Laboratories, Carlsbad, Calif.). 16S rRNA of V4 region was performed by the Second Genome (San Francisco, Calif.) using extracted DNA and V4-specific primers. Relative levels of Lactobacillus species were determined by quantitative real-rime PCR using species-specific and pan-bacterial Tagman® probes. HPV status was determined using DNA from collected vaginal swabs and the Linear Array HPV Genotyping Test (Roche, Indianapolis, Ind.) following the manufacturers instructions to classify patients into Ctrl HPV− and Ctrl HPV+ groups. Demographic data was collected from surveys and/or medical records. Statistical differences in the demographic variables between patient groups were tested using an analysis of variance (ANOVA) for continuous variables and Fisher's exact test for categorical variables. All statistical analyses were performed using SAS 9.4 (SAS Institute, Cary, N.C.), unless otherwise stated

Table 1 below shows the statistical significance in patient demographics between groups. P values were calculated using ANOVA for continuous variables and Fisher's exact test for categorical values. Ctrl HPV−: healthy HPV-negative control; Ctrl HPV+: healthy HPV− positive control; LSIL: low-grade squamous intraepithelial lesion; HSIL: low-grade squamous intraepithelial lesion; ICC: invasive cervical carcinoma.

TABLE 1

| | | Ctrl HPV− | | Ctrl HPV+ | | LSIL | | HSIL | | ICC | | P |
|---|---|---|---|---|---|---|---|---|---|---|---|---|
| | n | (n = 18) | | (n = 11) | | (n = 12) | | (n = 27) | | (n = 10) | | value |
| Age (mean (SD)) | 78 | 40.38 | (6.98) | 36.36 | (9.53) | 35.08 | (7.24) | 38.29 | (8.46) | 38.90 | (9.09) | 0.46 |
| Ethnicity (n (%)) | | | | | | | | | | | | |
| Hispanic | 37 | 5 | (27.78) | 4 | (36.36) | 7 | (58.33) | 17 | (62.96) | 4 | (40.00) | 0.15 |
| Non-Hispanic | 41 | 13 | (72.72) | 7 | (63.64) | 5 | (41.67) | 10 | (37.04) | 6 | (60.00) | |
| pH (n (%)) | | | | | | | | | | | | |
| ≤4.5 | 15 | 9 | (50.00) | 1 | (11.11) | 3 | (27.27) | 2 | (7.41) | 0 | (0.00) | |
| >4.5 | 59 | 9 | (50.00) | 8 | (88.89) | 8 | (72.73) | 25 | (92.59) | 9 | (100.00) | 0.003 |
| BMI (n (%)) | | | | | | | | | | | | |
| ≤25 | 25 | 7 | (38.89) | 3 | (27.27) | 4 | (33.33) | 8 | (29.63) | 3 | (30.00) | |
| >25 | 53 | 11 | (61.11) | 8 | (72.73) | 8 | (66.67) | 19 | (70.37) | 7 | (70.00) | 0.97 |
| HPV status | | | | | | | | | | | | |
| HPV 16 Positive | 43 | | | 9 | (81.82) | 8 | (72.73) | 19 | (70.37) | 7 | (70.00) | 0.78 |
| HPV 18 positive | 6 | | | 0 | (0.00) | 1 | (9.09) | 4 | (14.81) | 1 | (10.00) | 0.62 |
| Other high-risk | 38 | | | 5 | (45.55) | 10 | (83.33) | 19 | (70.37) | 4 | (40.00) | 0.09 |
| Low -risk HPV | 2 | | | 0 | (0.00) | 0 | (0.00) | 1 | (3.70) | 1 | (10.00) | 0.45 |
| HPV risk profile | | | | | | | | | | | | |
| Single high risk | 31 | | | 8 | (72.73) | 3 | (25.00) | 13 | (48.15) | 7 | (70.00) | 0.08 |
| Multiple high risk | 26 | | | 3 | (27.27) | 8 | (66.67) | 13 | (48.15) | 2 | (20.00) | 0.11 |
| High and low risk | 57 | | | 11 | (100.00) | 11 | (91.67) | 26 | (96.30) | 9 | (90.00) | 0.55 |

### Local Immune Checkpoint Proteins in Cervicovaginal Lavages:

Measurement of local immune checkpoint proteins: Levels of sixteen immune checkpoint proteins (BTLA, CD27, CD28, CD40, CD80/B7-1, CD86/B7-2, CTLA-4, GITR, GITRL. HVEM, ICOS, LAG-3, PD-1, PD-L1, TIM-3, TLR2) were determined in CVL samples using the MILLIPLEX MAP® Human Immuno-Oncology Checkpoint Protein Magnetic Bead Panel (Millipore, Bilerica, Mass.) in accordance with the manufacturer's protocol. Data was collected with a Bio-Plex® 200 instrument and analyzed using Manager 5.0 software (Blo-Rad, Hercules. Calif.). A four-parameter logistic regression curve fit was used to determine the concentration. All samples were assayed in duplicate. The concentration values below the detection limit were substituted with 0.5 of the minimum detectable concentration provided in manufacturer's instructions. The natural logarithm (in) transformation was applied to normalize the data.

Principal component analysis (PCA): The PCA was performed to reduce the observed variables into a smaller number of principal components (artificial variables) that will account for most of the variance in the observed variables. For the first two components, the difference among groups was assessed using the multivariate analysis of variance (MANOVA) model, if the overall difference was significant (P<0.05), pairwise comparisons with Tukey adjustment were performed. The statistical differences for individual components were assessed using ANOVA.

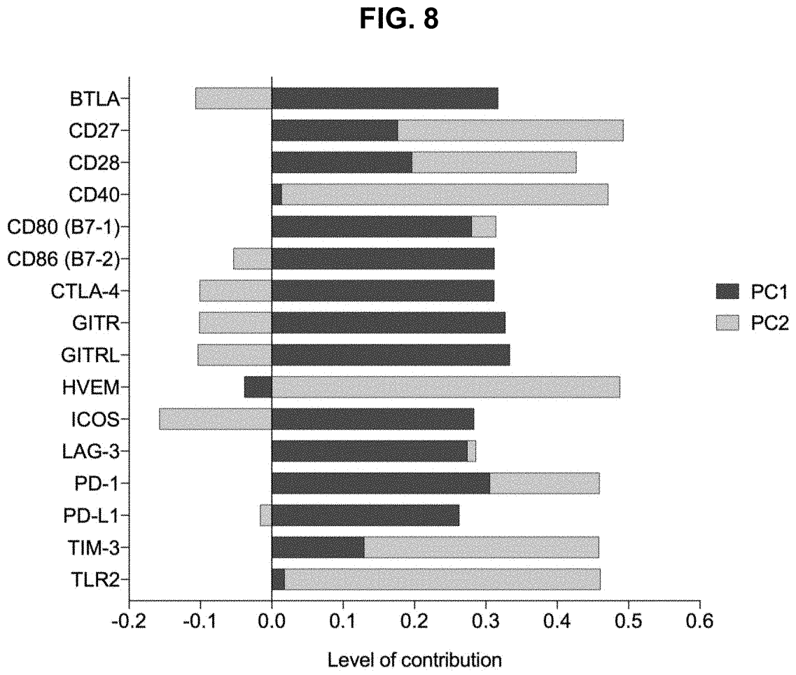

To investigate immune checkpoint protein profiles in the local cervicovaginal microenvironment, levels of 16 immune checkpoint proteins were measured in CVL samples collected from women across cervical carcinogenesis. AN protein targets were able to be detected. The principal component analysis (PCA), a data reduction method, was used to illustrate global immune checkpoint profiles among the groups (FIG. 1). The first two principal components (PC1 and PC2) utilized explained 70.4% of the variance in the data. Contributions of each immune checkpoint protein to PC1 and PC2 are shown in FIG. 8. Multivariate analysis of variance (MANOVA) revealed significant differences among the groups (P<0.0001). Subsequent pairwise comparisons showed that the ICC group is significantly different from all of the dysplasia (HSIL and LSIL) and control (Ctrl HPV− and Ctrl HPV+) groups (P ranging from 0.003 to 0.0001). Furthermore, when analyzing principal components separately, PC2, but not PC1, significantly varied between the ICC group when compared to all of the other groups (P ranging between 0.01 and <0.0001). This analysis demonstrated that immune checkpoint proteins can be detected in the local cervicovaginal microenvironment and ICC patients exhibit distinct immune checkpoint profiles compared to healthy HPV-negative women, HPV-positive women and women with precancerous dysplasia.

### Immune Checkpoint Proteins and Invasive Cervical Carcinoma:

Receiver operating characteristics analysis: The ROC analysis was performed to identify immune checkpoint proteins that discriminate specific patient groups. The mean levels of immune checkpoint proteins for each patient were used in the analyses. The strength of the discriminators was measured with area under the curve (AUC) values. Proteins with AUC greater than 0.7, 0.8 or 0.9 were considered as fair, good or excellent discriminators, respectively. The analysis was performed using Prism 5.0 software (GraphPad, San Diego, Calif.).

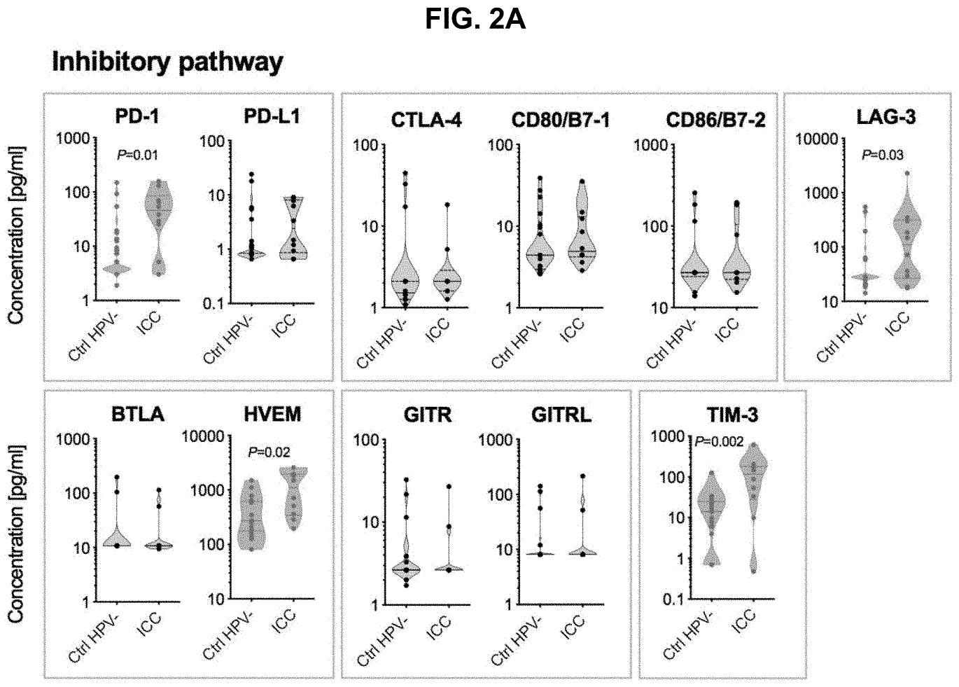

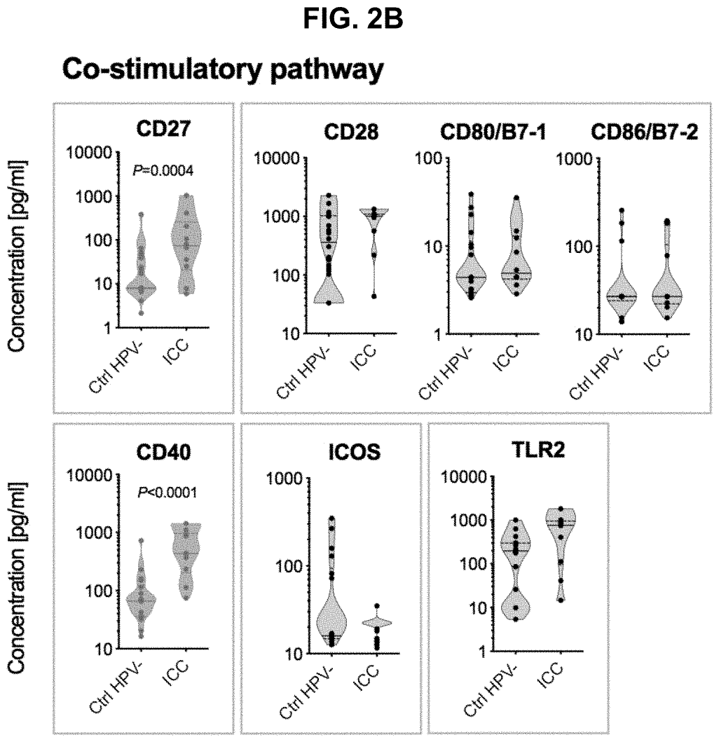

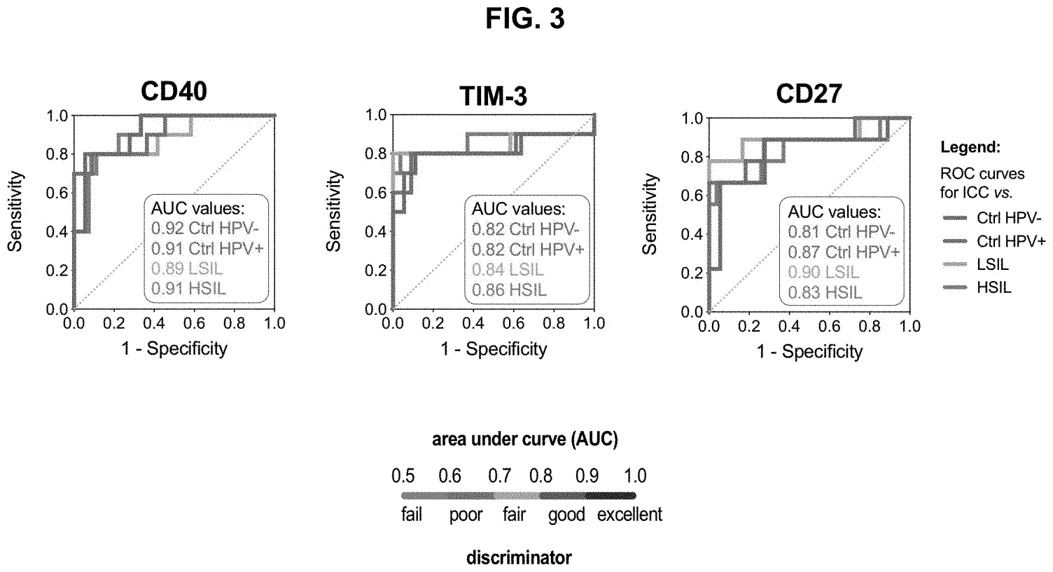

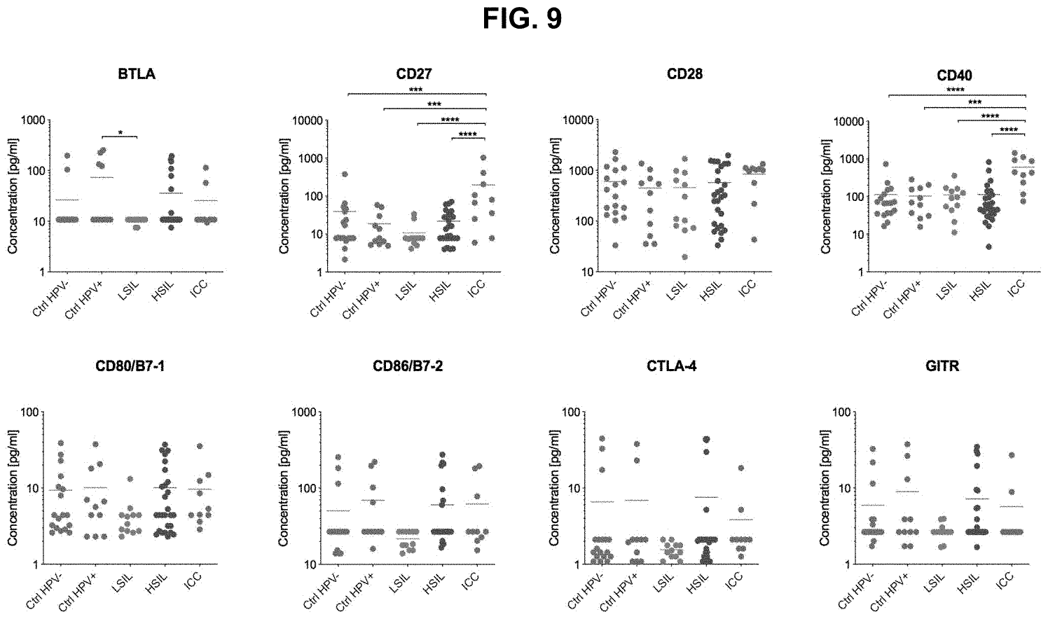

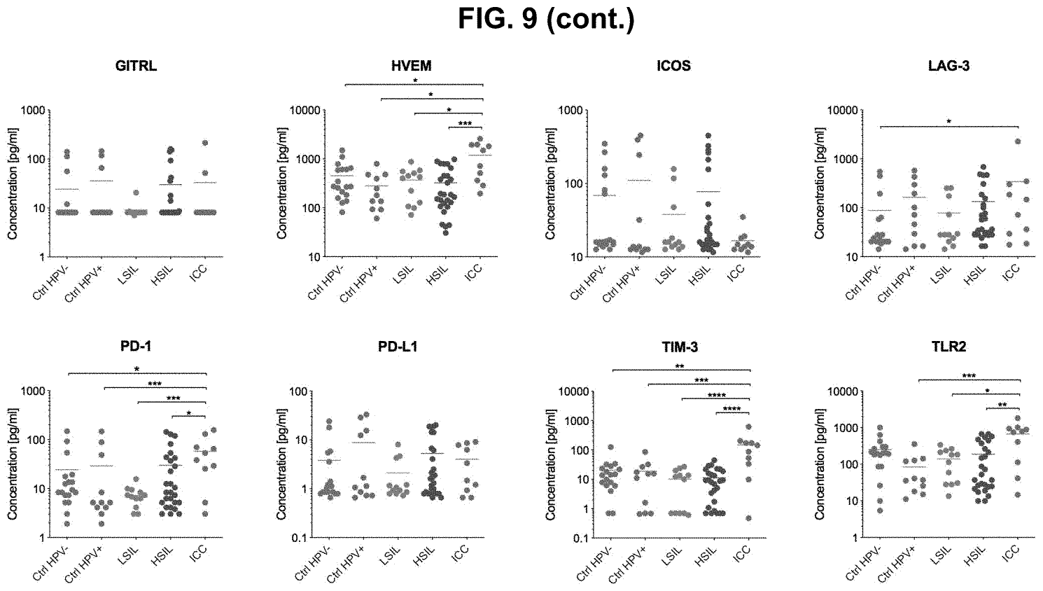

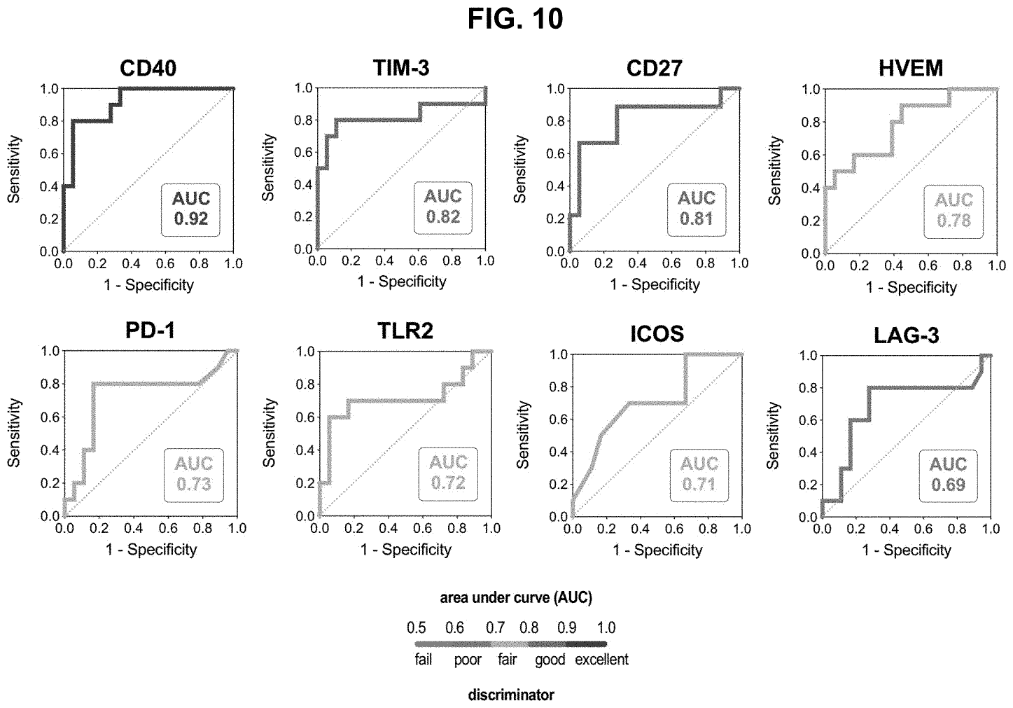

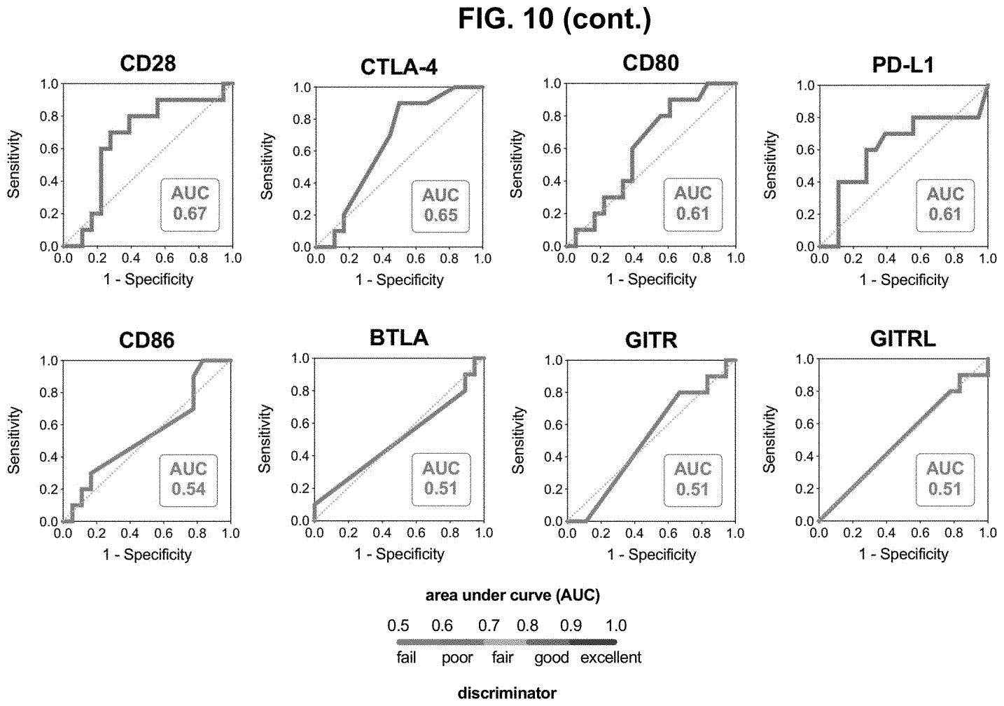

When the levels of immune checkpoint proteins measured in CVL samples were_compared among the groups, six out of sixteen targets were significantly elevated in women with ICC compared to the Ctrl HPV− group (P ranging from 0.03 to <0.0001) (FIG. 2) and other pre-cancerous groups (FIG. 9). Functionally, four immune checkpoint proteins, such as programmed cell death protein 1 (PD-1), lymphocyte activation gene 3 (LAG-3), herpesvirus entry mediator (HVEM) and T-cell immunoglobulin and mucin domain-containing 3 (TIM-3), are involved in the inhibitory pathways and two proteins, cluster of differentiation (CD) 27 and CD40, are involved in the co-stiliatory pathways. ICC patients also exhibited elevated levels of Toll-like receptor 2 (TLR2) when compared to dysplasia and Ctrl HPV+ groups (P ranging from 0.03 to 0.003), but not when compared to Ctrl HPV− (FIG. 9). None of the immune checkpoint proteins were significantly elevated in dysplasia groups when compared to controls (FIG. 9). A receiver operating characteristic (ROC) curve analysis was also performed to evaluate the discrimination capacity of tested immune checkpoint proteins (FIG. 3 and FIG. 10). Proteins with area under the curve (AUC), which plots the true positive rate (sensitivity) against the false positive rate (1—specificity), greeter than 0.9 or 0.8 were considered as excellent or good discriminators, respectively. The analysis comparing ICC and Ctrl HPV− groups revealed three immune checkpoint proteins with excellent or good discriminatory properties, such as CD40 (AUC 0.92), TIM-3 (AUC 0.82) and CD27 (AUC 0.81). HVEM, PD-1, TLR2 and inducible T-cell co-stimulator (ICOS) exhibit only fair discrimination capabilities (AUC>0.7), whereas other immune checkpoint proteins were poor discriminators (AUC<0.7) (FIG. 10). When comparing other groups, CD40, TIM-3 and CD27 also significantly discriminated ICC group from dysplasia and Ctrl HPV+ groups (AUC ranging from 0.82 to 0.91). These analyses demonstrated that specific immune checkpoint proteins are significantly and specifically elevated in women with ICC.

### Cervicovaginal Immune Checkpoint Protein Correlation Network

Correlation analyses: The Spearman's rank correlation analyses were performed to investigate association of immune checkpoint proteins to other immune checkpoint proteins, vaginal microbiota and genital inflammation. Spearman's rank correlation coefficients (ρ) were computed using log-transformed levels of immune checkpoint proteins among all patients (n=78), relative abundance of vaginal bacterial genera (i.e., Lactobacillus, Gardnerella, Prevotella, Sneathia, Atopobium, Megasphera, Strepkococcus), relative levels of vaginal Lactobacillus species (L. crispatus, L. gasseri, L. jensenii, L. iners) and genital inflammatory scores. Relative abundances of vaginal taxa were determined by 16S rRNA gene sequencing and relative levels of Lactobacillus species were determined by quantitative real-rime PCR using species-specific and pan-bacterial Taqman® probes as described below. Genital inflammatory scoring was also described previously in Laniewski, P et al. 2018 (Laniewski, P. et al. Unking cervicovaginal immune sgnatures, HPV and microbiota composition in cervical carcinogenesis in non-Hispanic and Hispanic women. Sol Rep 8, 7593, doi:10.1038/s41598-018-25879-7, 2018). Briefly, levels of seven cytokines (IL-1α, IL-1β, IL-8, MIP-10, MIP-3a/CCL20, RANTES, TNFα) were evaluated in CVLs and the patients were assigned a genital inflammatory score (0-7) based on whether the level of each cytokine was in the upper quartile. P values<0.05 were considered significant.

Quantitative real-time PCR analysis: Relative abundance of four LactobacOlus spp. was determined by quantitative real-time PCR analysis, performed on an Applied Biosystems QuantStudio6 Flex Real Time PCR System (Life Technologies, Grand Island, N.Y.) using DNA extracted from vaginal swabs, TaqMan Assays specific for L. crispatus, L. gasseri, L. iners, L. jensenii and panbacterial 166 rRNA genes and TaqMan Vaginal Microbiota Amplification Control and TaqMan Fast Advanced Master Mix (Life Technologies). Relative abundances were calculated using a standard curve method and the 16S rRNA gene level as an internal standard.

Hierarchical clustering analysis (HCA): The HCA was performed to show relationships of immune checkpoint proteins to other immune checkpoint proteins. Clustering of computed correlation coefficients (ρ) was performed using ClustVis and based on Euclidean distance between rows and columns and average linkage cluster algorithm as described in Metsalu T., et al. 2015 (Metsalu T, & Vilo, J. ClustVis: a web tool for visualizing clustering of multivariate data using Principal Component Analysis and heatmap. Nucleic Acids Res 43, W566-570, doi:10.1093/nar/gkv468 (2015))

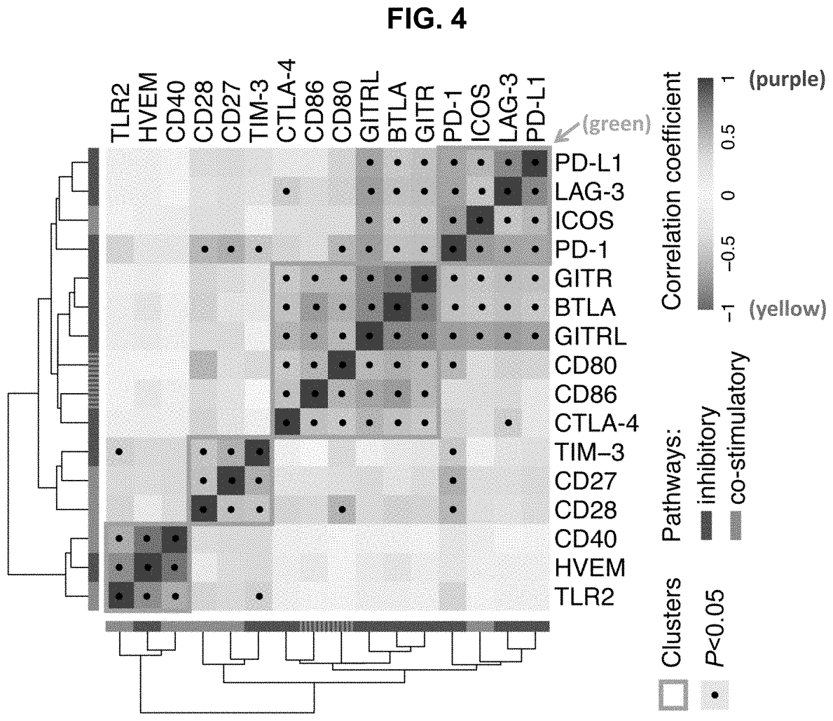

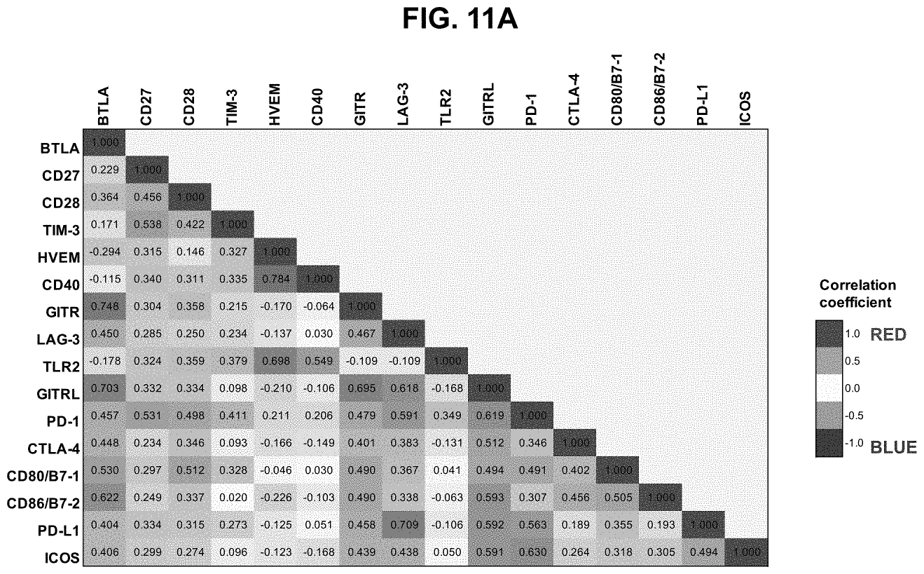

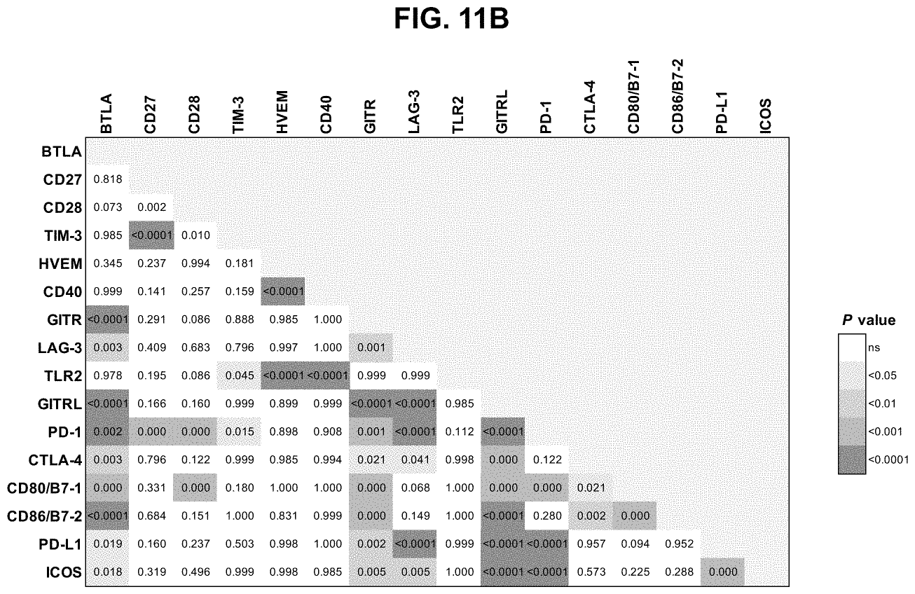

To identify potential biological networks among the immune checkpoint proteins, a correlation matrix was computed using the levels of immune checkpoints among all the patients regardless of the groups (FIGS. 11A-11B). Calculated Spearman's coefficients (ρ) for each pair of immune checkpoint proteins were used in the hierarchical clustering analysis (HCA) and depicted as a heatmap (FIG. 4). The HCA revealed four major dusters of immune checkpoint proteins that significantly and positively correlated to each other: cluster 1 with CD40, HVEM, TLR2; duster 2 with C027, C028, TIM-3; duster 3 with B- and T-lymphocyte attenuator (BTLA), CD80, CD88, CTLA-4, glucocorticoid-induced tumor necrosis factor receptor-related protein (GITR), GITR ligand (GITRL); and duster 4 with ICOS, LAG-3, PD-1, programmed cell death ligand 1 (PD-L1). All four dusters comprised of a mix of immune checkpoints belonging to both inhibitory and co-stimulatory pathways. Furthermore, the levels of immune checkpoint molecules in CVL samples positively and significantly correlated to the levels of their ligands, e.g. PD-1 levels correlated to PD-L1 levels (ρ=0.523, P<0.0001), CTLA-4 to CD80 (ρ=0.402, P=0.021) and CD88 (ρ=0.458, P=0.002), GITR to GITRL (ρ=0.695, P<0.0001). These data suggest that the production and/or secretion of immune checkpoint proteins is co-dependent, and these molecules form a complex biological network involving both immune inhibitory and immune co-stimulatory pathways.

### Immune Checkpoint Proteins, Inflammation, and Microbiota

16S rRNA sequencing analysis: 16S rRNA sequencing analysis was performed by the Second Genome Inc. (San Francisco, Calif.). Briefly, the V4 region of bacterial 16S rRNA operon was amplified from the genomic DNA obtained from vaginal swabs and sequenced on the MiSeq platform (Illumina, San Diego, Calif.). The samples were analyzed using USEARCH (for details see Laniewski, P et al. 2018 (Laniewski, P. et a. Linking cervicovaginal immune signatures, HPV and microbiota composition in cervical carcinogenesis in non-Hispanic and Hispanic women. Sci Rep 8, 7593, doi:10.1038/s41596-018-25879-7, 2018).

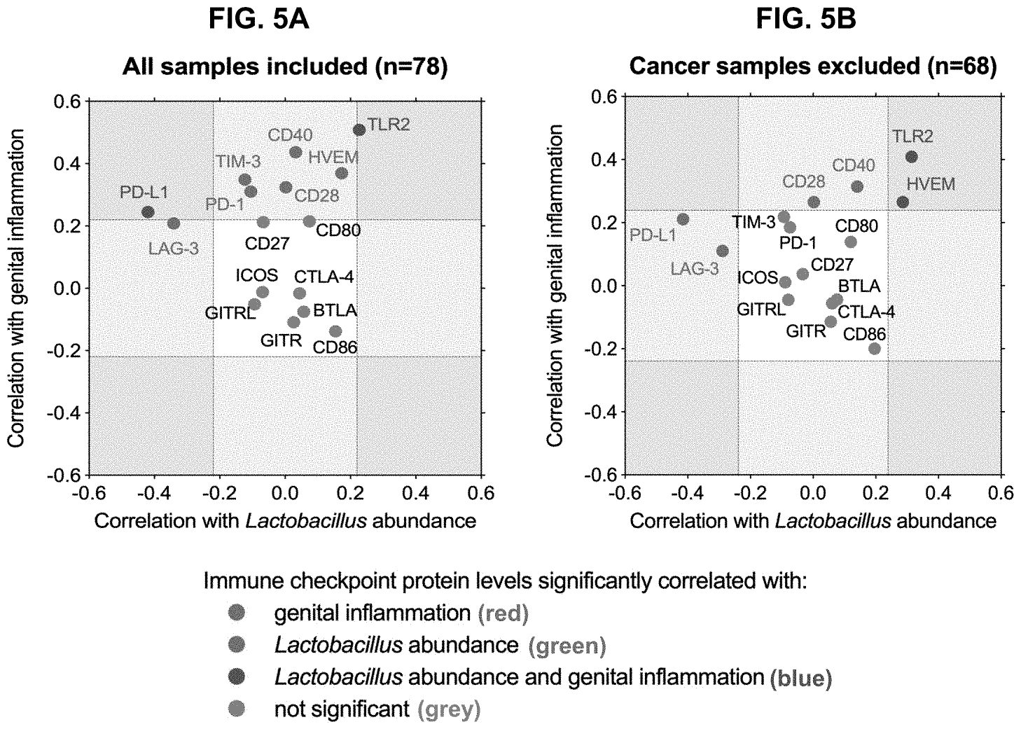

The severity of cervical neoplasia is linked to genital inflammation and vaginal microbiota composition. The relationships between the levels of immune checkpoint proteins, genital inflammatory scores and Lactobacillus abundance were investigated. The genital inflammation scoring system was described previously described in Laniewski, P et al. 2018 (Laniewski, P. of a. Linking cervicovaginal immune signatures, HPV and microbiota composition in cervical carcinogenesis in non-Hispanic and Hispanic women. Sci Rep 8, 7593. doi:10.1038/s41598-018-25879-7, 2018). Briefly, levels of seven cytokines, including interleukin (IL)-1α, IL-1β, IL-8, macrophage inflammatory protein (MIP)-1β, MIP-3α, regulated on activation, normal T cell expressed and secreted (RANTES), and tumor necrosis factor (TNF), were evaluated in CVL samples and women were assigned a genital inflammatory score (0-7) based on whether the level of each cytokine was in the upper quartile. These cumulative inflammatory scores also reflected elevated levels of other pro-inflammatory immune mediators tested, but not included in the score, therefore this scoring system can accurately reflect genital inflammation. Relative abundance of Lactobacillus and other vaginal genera was determined previously using 16S rRNA gene sequencing and DNA extracted from vaginal swabs. The Spearman's correlation coefficients (ρ) were calculated for each immune checkpoint protein (in all patients regardless of disease group) and genital inflammatory scores, as well as, for each immune checkpoint protein and Lactobacillus abundance. The computed correlation coefficients were depicted as a scatterplot showing the correlation with inflammatory scores on y axis and correlation with Lactobacillus abundance on x axis (FIG. 5A). The analysis demonstrated that several immune checkpoint proteins (CD28, CD40, HVEM, PD-1, PD-L1, TIM-3, TLR2) positively (ρ ranging from 0.245 to 0.508) and significantly (P ranging from 0.03 to <0.001) correlated to genital inflammation. Two of these immune checkpoint proteins also correlated to Lactobacillus abundance: PD-L1 in a negative relationship (ρ=−0.420; P<0.001) and TLR2 in a positive relationship (ρ=0.227; P=0.048). However, LAG-3 negatively correlated to Lactobacillus abundance (ρ=−0.341; P=0.002) but did not significantly correlate to genital inflammation.

To better understand the relationships independent of cancer, a correlation analysis was also performed by excluding ICC samples (FIG. 51). In this analysis, CD28 (ρ=0.265; P=0.029) and CD40 (ρ=0.314; P=0,009) still positively correlated to genital inflammation, whereas HVEM and TLR2 positively correlated to both genital inflammation and Lactobacillus abundance (ρ ranging from 0.265 to 0.409; P ranging from 0.029 to 0.001). PD-L1 did not correlate to genital inflammation; however, it still exhibited a negative correlation to Lactobacillus abundance (ρ=−0.415; P=0.001), similarly to LAG-3 (ρ=−0.289; P=0.018). These analyses revealed associations between the local levels of several immune checkpoint proteins with the vaginal microbiota composition and genital inflammation, which was independent of cancer.

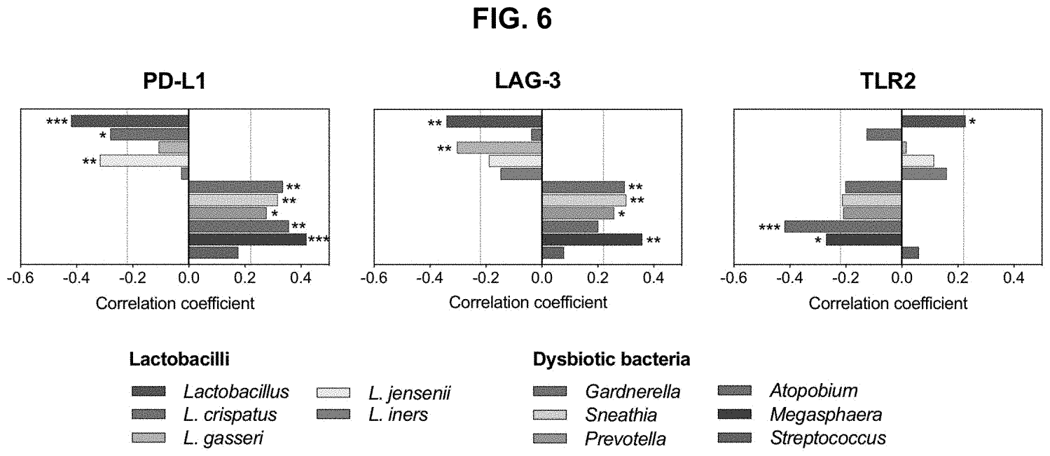

) To further explore the relationship between PD-L1, LAG-3 and TLR2 and vaginal microbiota composition, additional correlation analyses were performed using the relative abundance of the most prevalent vaginal bacterial taxa detected in the samples, including four predominant vaginal LactobaciNus species (L. crispatus, L. gasser, L. jensenii and L. iners), as well as, bacteria associated with vaginal dysbiosis (Gardnerella, Sneathia, Prevotella, Atopobium, Megasphaera) and vaginal pathobionts (Streptococcus) (FIG. 6). The analysis revealed significant correlations between the PD-L1, LAG-3, TLR2 levels and abundance/levels of multiple vaginal bacterial taxa. The directionality of these correlations varied for health-associated Lactobacillus species versus bacteria associated with vaginal dysbiosis for each of the immune checkpoint proteins. For instance, PD-L1 negatively correlated to L. crispatus (ρ=−0.280; P=0.014) and L. jensenii (ρ=−0.317; P=0.005) and positively correlated dysbiosis-associated bacteria Gardnerella, Sneathia, Prevotella, Atopobium and Megasphaera (ρ ranging from 0.278 to 0.420; P ranging from 0.015 to 0.001), whereas LAG-3 negatively correlated to L. gasseri (ρ=−0.304; P=0.007) and positively correlated to Gardnerella, Sneathia, Prevotella and Megasphaera (ρ ranging from 0.259 to 0.359; P ranging from 0.023 to 0.001). On the other hand, TLR2 positively correlated to Lactobacillus (ρ=0.227; P=0.046), and negatively correlated to Atopobium (ρ=−0.420; P<0.001) and Megasphaera (ρ=−0.271; P=0.017). However, with TLR2 no significant correlations were observed to specific vaginal Lactobacillus species. Additionally, no significant correlations of PD-L1, LAG-3 or TLR2 to Streptococcus (vaginal pathobiont) or L. iners (intermediate Lactobacillus species associated with the transition to vaginal dysbiosis) were observed. This analysis further confirmed the observed strong associations (FIG. 5) between the local levels of immune checkpoint proteins and the vaginal microbiota composition

In this study, a broad range of immune checkpoint proteins were examined in the local cervicovaginal microenvironment in women with and without cervical neoplasm and the relationships among these key immunoregulation proteins, the vaginal microbiota composition and genital inflammation, were explored. The present invention demonstrates that immune checkpoint proteins can be measured in cervicovaginal lavages (CVL), which indicate the presence of soluble forms of these proteins in the cervicovaginal microenvironment. To date, expression of immune checkpoint proteins in cervical cancer patients have been mostly evaluated in biopsied tissue samples using immunohistochemical staining. Compared to biopsy, the collection of CVL is a minimally invasive, low cost, and relatively easy procedure to perform. Furthermore, protein detection in CV has a great potential to be exploited for monitoring responses to therapies, as well as used as predictive or prognostic biomarkers in women with cervical cancers and other gynecological conditions.

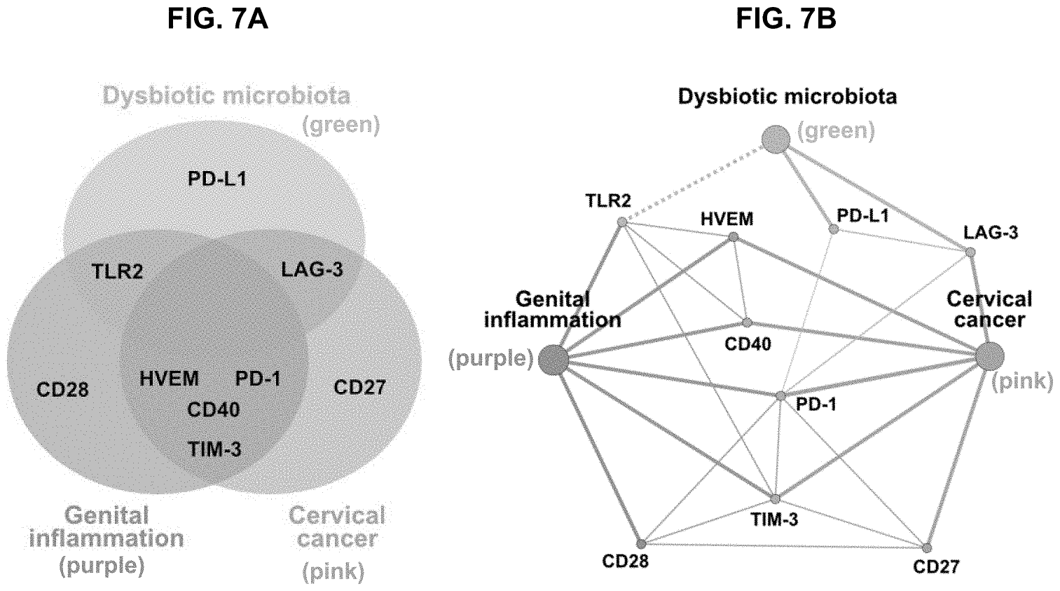

In summary, the present invention has identified immune checkpoint signatures associated with cervical carcinogenesis and illuminated the multifaceted microbiota-host immunity network in the local microenvironment (FIGS. 7A-8). Elevated levels of CD40, HVEM, PD-1 and TIM-3 connected cervical carcinoma to genital inflammation, whereas LAG-3 connected carcinoma to dysbiotic microbiota and TLR2 bridged genital inflammation and Lactobacillus dominance. None of the immune checkpoint proteins tested related to al features of cancer, inflammation and microbiota; however, multiple immune checkpoint proteins correlated to each other, relating all features together, which highlight the complex interactions between host, HPV and microbiota during cervical carcinogenesis.

In conclusion, immune checkpoint molecules can be detected in the cervicovaginal microenvironment in women across cervical carcinogenesis and, notably, that the levels of these molecules depend on genital inflammation and the vaginal microbiota composition. In the future, these or other protein targets, measured in cervicovaginal lavages, might be utilized as prognostic or predictive biomarkers for cervical cancer and other gynecologic conditions. These relationships in the cervicovaginal microenvironment, similarly to those observed in the gut, may impact responsiveness to immunotherapy and/or immune-related toxicities, particularly in patients with gynecologic cancer, and should be explored in future clinical studies.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A method of diagnosing invasive cervical carcinoma (ICC) in a patient, the method comprising:
a) determining the patient's levels of two or more immune checkpoint proteins by:
i) obtaining a cervicovaginal lavage (CVL) sample from the patient;
ii) measuring the levels of two or more checkpoint proteins in the sample obtained in (1); and
b) if the patient has levels of at least two or more immune checkpoint proteins above a predetermined threshold then the patient is diagnosed with ICC or if the patient has levels of at least two or more immune checkpoint proteins below a predetermined threshold then the patient is diagnosed with dysplasia;
wherein the predetermined threshold is the immune biomarker checkpoint protein concentration over a defined threshold or fold change, or specific concentration in pg/ml.

2. The method of claim 1, wherein the immune checkpoint protein is a duster of differentiation 40 (CD40).

3. The method of claim 2, wherein the level of CD40 above 200 pg/ml is indicative of ICC.

4. The method of claim 1, wherein the immune checkpoint protein is a duster of differentiation 27 (CD27).

5. The method of claim 4, wherein the level CD27 above 20 pg/ml is indicative of ICC.

6. The method of claim 1, wherein the immune checkpoint protein is a T-cell immunoglobuin and mucin domain-containing 3 (TIM-3).

7. The method of claim 6, wherein the level of TIM-3 above 20 pg/ml is indicative of ICC.

8. A method of predicting a response to a therapy for treating invasive cervical carcinoma (ICC), the method comprises:
a) obtaining a cervicovaginal lavage (CVL) sample from a patient
b) analysing said sample to detect levels of at least two biomarkers selected from a group consisting of duster of differentiation (CD) 40, T-cell immunoglobulin and mucin domain-containing 3 (TIM-3), CD27, programmed cell death protein ligand 1 (PD-L1), lymphocyte activation gene 3 (LAG-3), toll-like receptor 2 (TLR-2), herpesvirus entry mediator (HVEM), CD28, cytotoxic T-lymphocyte antigen 4 (CTLA-4), glucocorticoid-induced tumor necrosis factor receptor-related protein (GITR), GITR ligand (GITRL), CD86, B- and T-lymphocyte attenuator (BTLA), inducible T-cell co-stimulator (ICOS), CD80, Lactobacillus abundance, and inflammation,
wherein the levels of the at least two biomarkers are indicative of a particular state of invasive cervical carcinoma and indicate whether the response to a therapy will be positive or negative.

9. The method of claim 8, wherein a positive response is a regression of cancer.

10. The method of claim 8, wherein a negative response is a progression of cancer.

11. The method of claim 8, wherein the level of CD40 above 200 pg/ml is indicative of ICC.

12. The method of claim 8, wherein the level of CD27 above 20 pg/ml is indicative of ICC.

13. The method of claim 8, wherein the level of TIM-3 above 20 pg/ml is indicative of ICC.

14. The method of claim 8, wherein the method predicts toxicity in a patient in response to a therapy.

15. The method of claim 8, wherein the method can stratify patients in a cohort into a group of responders and non-responders.

16. The method of claim 15, wherein responders are patients predicted to have a positive response to a therapy for treating ICC.

17. The method of claim 15, wherein non-responders are patients predicted to have no response or a negative response to a therapy for treating ICC.

18.-21. (canceled)

22. A method comprising:
a) obtaining a cervicovaginal lavage (CVL) sample from a patient
b) producing a profile of the CVL sample collected in (a) by:
i) detecting at least two or more immune checkpoint biomarkers, and
ii) detecting the microbiota population
c) analysing the CVL sample profile produced in (b).
